(19) United States
(12) Patent Application Publication
(10) Pub. No.: US 2021/0346183 A1
(43) Pub. Date: Nov. 11, 2021

(54) BARIATRIC BALLOON SYSTEMS

(71) Applicant: ARIZONA BOARD OF REGENTS ON BEHALF OF THE UNIVERSITY OF ARIZONA, Tucson, AZ (US)

(72) Inventor: Seda Bourikian, Phoenix, AZ (US)

(21) Appl. No.: 17/283,876

(22) PCT Filed: Oct. 9, 2019
(86) PCT No.: PCT/US19/55364

§ 371 Date: Apr. 8, 2021

Related U.S. Application Data
(60) Provisional application No. 62/743,348, filed on Oct. 9, 2018.

(51) Int. Cl. A61F 5/00

(52) U.S. Cl. CPC A61F 5/0033; A61F 5/0043

(57) ABSTRACT

A bariatric balloon system for reducing the volume of the stomach, featuring a plurality of balloons each fluidly connected to a tube with a tube port at its end. The system is secured to the abdominal wall. The system can be implanted such that the port is outside the skin or is under the skin. The tube extends through the skin and/or muscle and fat to the stomach wherein the balloons are placed. The balloons can be inflated by introducing fluid such as saline to the tube via the port.

### CROSS-REFERENCES TO RELATED APPLICATIONS

This application is a PCT and claims benefit of U.S. Patent Application No. 62/743,348, filed Oct. 9, 2018, the specification(s) of which is/are incorporated herein in their entirety by reference.

### BACKGROUND OF THE INVENTION

### Field of the Invention

The present invention relates to weight loss, more particularly to bariatric balloons for inserting into the stomach to help reduce the available volume of the stomach.

### Background Art

Apart from diet and exercise, weight loss can be achieved in humans by reducing the effective volume of the stomach, thereby reducing the amount of food consumed. Various approaches have been used to reduce the volume of the stomach. For example, one approach is to surgically remove part of the stomach; other approaches include reconstructing the pathways of the GI tract. These methods have benefits, but also have associated risks because of the significant amount of permanent and invasive alterations made to the patient's anatomy, and complications from these surgeries can be devastating.

Another approach to weight loss is the short-term use of a free-floating balloon, which allows one to feel satiated quicker by reducing the available volume in the stomach. However, the free-floating balloons also pose risks, as collapse of the balloon can lead to obstruction. Further, the free-floating balloons are tailored for a lower BMI population.

### BRIEF SUMMARY OF THE INVENTION

The present invention features bariatric balloon systems for insertion into the stomach. The balloon system of the present invention comprises a tube that is inserted through and into the stomach and a plurality of balloons (e.g., two, three, four, etc.), each connected to the tube. The system is secured to the abdominal wall. The volume of the balloons is changed by filling them with a liquid (e.g., saline) via the port at the end of the tube. The balloons may be designed to fill sequentially. As an example, in certain embodiments, the balloons may be engineered to automatically fill sequentially. In certain embodiments, the port is separated into different sub-ports, e.g., one sub-port for each balloon, wherein the balloons can be filled sequentially manually. For example, a system may feature three balloons each with their own port or sub-port, wherein a first balloon is filled first, then a subsequent balloon, and then the final balloon.

The present invention is not limited to sequential filling. For example, in certain embodiments, two or more balloons may fill up simultaneously. In certain embodiments, the first balloon, e.g., the balloon closes to the port, fills first and then two or more of the remaining balloons fill simultaneously.

In some embodiments, the port is secured externally, e.g., to the skin, to allow for the patient to control the balloon size. In some embodiments, the port is secured under the skin, e.g., under the skin and over the fascia. With the port secured under the skin, a physician or other healthcare provider may be required to alter the balloon size.

Without wishing to limit the present invention to any theory or mechanism, it is believed that the system of the present invention is advantageous because the system is less likely to lead to obstruction compared to free-floating balloons. For example, the system of the present invention is secured in place via the port (e.g., outside the skin or under subcutaneous tissue). If a free-floating balloon becomes deflated, it can slide to the intestine, leading to obstruction. In contrast, because the system of the present invention is anchored in place via the port, it would not slide to the intestine in a deflated state. Further, accessibility of the port outside the skin can give the patient the freedom to control the volume of the balloons. Or, if the patient would prefer that the port not be visible over the skin, the port can be secured under subcutaneous tissue. The port can still be accessed if secured subcutaneously, however a needle or other device that can penetrate to the port would be required.

Still further, the present invention provides patients a temporary treatment option if they are not willing to undergo significant and permanent surgery. The system of the present invention may be able to be implanted endoscopically or by interventional radiologists, allowing the patient to have a procedure under lighter anesthesia as compared to traditional bariatric surgeries.

In certain embodiments, the bariatric balloon system of the present invention, for reducing the volume of the stomach, comprises a tube having a port at a first end; a first balloon fluidly connected to the tube; and a second balloon fluidly connected to the tube. The first balloon and the second balloon are inflatable by inserting fluid into the tube via the port.

In certain embodiments, the system further comprises a third balloon fluidly connected to the tube, the third balloon is inflatable by inserting fluid into the tube via the port. In certain embodiments, the system further comprises a fourth balloon fluidly connected to the tube, the fourth balloon is inflatable by inserting fluid into the tube via the port. In certain embodiments, the system further comprises a fifth balloon fluidly connected to the tube, the fifth balloon is inflatable by inserting fluid into the tube via the port. In certain embodiments, the system further comprises a sixth balloon fluidly connected to the tube, the sixth balloon is inflatable by inserting fluid into the tube via the port.

In certain embodiments, the second balloon is below the first balloon along the tube. In certain embodiments, the third balloon is below the second balloon along the tube. In certain embodiments, the fourth balloon is below the third balloon along the tube. In certain embodiments, the fifth balloon is below the fourth balloon along the tube. In certain embodiments, the sixth balloon is below the fifth balloon along the tube.

In certain embodiments, the first balloon reaches its maximum volume before the second balloon reaches its maximum volume. In certain embodiments, the second balloon reaches its maximum volume before the third balloon reaches its maximum volume. In certain embodiments, the third balloon reaches its maximum volume before the fourth balloon reaches its maximum volume. In certain embodiments, the fourth balloon reaches its maximum volume before the fifth balloon reaches its maximum volume. In certain embodiments, the fifth balloon reaches its maximum volume before the sixth balloon reaches its maximum volume.

In certain embodiments, the first balloon has a maximum volume larger than that of the second balloon. In certain embodiments, the first balloon has a maximum volume larger than that of the third balloon. In certain embodiments, the first balloon has a maximum volume larger than that of the fourth balloon. In certain embodiments, the first balloon has a maximum volume larger than that of the fifth balloon. In certain embodiments, the first balloon has a maximum volume larger than that of the sixth balloon. In certain embodiments, the second balloon has a maximum volume larger than that of the third balloon. In certain embodiments, the third balloon has a maximum volume larger than that of the fourth balloon. In certain embodiments, the fourth balloon has a maximum volume larger than that of the fifth balloon. In certain embodiments, the fifth balloon has a maximum volume larger than that of the sixth balloon.

In certain embodiments, the bariatric balloon system comprises: a tube having a port at a first end; a first balloon fluidly connected to the tube; a second balloon fluidly connected to the tube; and a third balloon fluidly connected to the tube (120). The first balloon, the second balloon, and the third balloon are inflatable by inserting fluid into the tube via the port.

In certain embodiments, the system further comprises a fourth balloon fluidly connected to the tube, the fourth balloon is inflatable by inserting fluid into the tube via the port. In certain embodiments, the system further comprises a fifth balloon fluidly connected to the tube, the fifth balloon is inflatable by inserting fluid into the tube via the port. In certain embodiments, the system further comprises a sixth balloon fluidly connected to the tube, the sixth balloon is inflatable by inserting fluid into the tube via the port.

In certain embodiments, the second balloon is below the first balloon along the tube. In certain embodiments, the third balloon is below the second balloon along the tube. In certain embodiments, the fourth balloon is below the third balloon along the tube. In certain embodiments, the fifth balloon is below the fourth balloon along the tube. In certain embodiments, the sixth balloon is below the fifth balloon along the tube.

In certain embodiments, the first balloon reaches its maximum volume before the second balloon reaches its maximum volume. In certain embodiments, the second balloon reaches its maximum volume before the third balloon reaches its maximum volume. In certain embodiments, the third balloon reaches its maximum volume before the fourth balloon reaches its maximum volume. In certain embodiments, the fourth balloon reaches its maximum volume before the fifth balloon reaches its maximum volume. In certain embodiments, the fifth balloon reaches its maximum volume before the sixth balloon reaches its maximum volume.

In certain embodiments, the first balloon has a maximum volume larger than that of the second balloon. In certain embodiments, the first balloon has a maximum volume larger than that of the third balloon. In certain embodiments, the first balloon has a maximum volume larger than that of the fourth balloon. In certain embodiments, the first balloon has a maximum volume larger than that of the fifth balloon. In certain embodiments, the first balloon has a maximum volume larger than that of the sixth balloon. In certain embodiments, the second balloon has a maximum volume larger than that of the third balloon. In certain embodiments, the third balloon has a maximum volume larger than that of the fourth balloon. In certain embodiments, the fourth balloon has a maximum volume larger than that of the fifth balloon. In certain embodiments, the fifth balloon has a maximum volume larger than that of the sixth balloon.

In certain embodiments, the bariatric balloon system for reducing the volume of the stomach comprises a first tube having a first port at an external end of the first tube; a first balloon fluidly connected to the first tube; a second tube having a second port at an external end of the second tube; and a second balloon fluidly connected to the second tube. The first balloon is inflatable by inserting fluid into the first tube via the first port, and the second balloon is inflatable by inserting fluid into the second tube via the second port.

In certain embodiments, the system further comprises a third tube having a third port at an external end of the third tube, and a third balloon fluidly connected to the third tube, the third balloon is inflatable by inserting fluid into the third tube via the third port. In certain embodiments, the system further comprises a fourth tube having a fourth port at an external end of the fourth tube, and a fourth balloon fluidly connected to the fourth tube, the fourth balloon is inflatable by inserting fluid into the fourth tube via the fourth port. In certain embodiments, the system further comprises a fifth tube having a fifth port at an external end of the fifth tube, and a fifth balloon fluidly connected to the fifth tube, the fifth balloon is inflatable by inserting fluid into the fifth tube via the fifth port. In certain embodiments, the system further comprises a sixth tube having a sixth port at an external end of the sixth tube, and a sixth balloon fluidly connected to the sixth tube, the sixth balloon is inflatable by inserting fluid into the sixth tube via the sixth port.

In certain embodiments, the first balloon has a maximum volume larger than that of the second balloon. In certain embodiments, the second balloon has a maximum volume larger than that of the third balloon. In certain embodiments, the third balloon has a maximum volume larger than that of the fourth balloon. In certain embodiments, the fourth balloon has a maximum volume larger than that of the fifth balloon. In certain embodiments, the fifth balloon has a maximum volume larger than that of the sixth balloon.

In certain embodiments, the bariatric balloon system for reducing the volume of the stomach comprises: a tube having a port at a first end; and at least a first balloon and a last balloon, wherein the tube extends through the first balloon and through the last balloon. The first balloon is fluidly connected to the tube via a pore disposed in the tube at a location within the first balloon and the last balloon is fluidly connected to the tube via a pore disposed in the tube at a location within the last balloon. The first balloon and the last balloon are inflatable by inserting fluid into the tube via the port.

In certain embodiments, the system comprises a first balloon, a last balloon, and at least one additional balloon. In certain embodiments, the system comprises a first balloon, a last balloon, and at least two additional balloons. In certain embodiments, the system comprises a first balloon, a last balloon, and at least three additional balloons. In certain embodiments, the tube ends in the last balloon. In certain embodiments, the balloons fill sequentially.

With respect to any of the examples of the systems herein, in certain embodiments, the tube is flexible. In certain embodiments, at least a portion of the tube is flexible.

The present invention also features methods for reducing the volume of a stomach of a patient in need thereof. The method may comprise implanting a bariatric balloon system of the present invention. For example, the system may be positioned with the balloons in the stomach and the tube extending through at least the fascia tissue of the skin. In some embodiments, the port is secured under the skin. In some embodiments, the tube and port extend through and outside the skin. The port may be secured to the skin. In some embodiments, the system is sutured to the stomach. The balloons are inflatable by inserting fluid therein via the port, thereby reducing the volume of the stomach of the patient. The method also applies to a method of reducing body weight of a patient. Reducing the volume of the stomach of the patient may help reduce food intake, thereby reducing the body weight of the patient.

As discussed herein, in certain embodiments, the system comprises a first balloon, a last balloon, and at least one additional balloon. In some embodiments, the system comprises a first balloon, a last balloon, and at least two additional balloons. In some embodiments, the system comprises a first balloon, a last balloon, and at least three additional balloons. In some embodiments, the tube ends in the last balloon. In some embodiments, the balloons fill sequentially. The methods of the present invention are not limited to the aforementioned embodiments and may feature any of the embodiments described herein.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a detailed view of the system of the present invention.

FIG. 2A shows an in-use view of a system of the present invention, wherein the port of the system is external (e.g., outside of the skin).

FIG. 2B shows a detailed view of the system of FIG. 2A.

FIG. 3A shows an in-use view of a system of the present invention, wherein the port of the system is implanted subcutaneously (e.g., under skin, over fascia).

FIG. 3B shows a detailed view of the system of FIG. 3A.

### DETAILED DESCRIPTION OF THE INVENTION

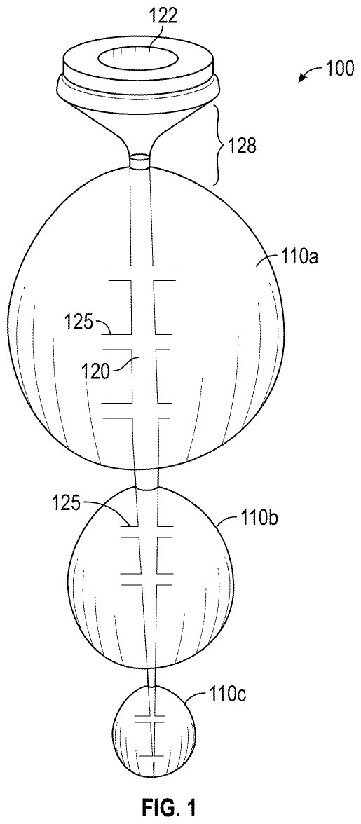

The present invention features a bariatric balloon system (100) for reducing the volume of the stomach (see FIG. 1). The bariatric balloon system (100) of the present invention is not a free-floating balloon. Briefly, the bariatric balloon system (100) herein features a plurality of balloons (110) each fluidly connected to a tube (120) with a tube port (122) at the end of the neck (128) of the tube (120). In certain embodiments, each balloon is fluidly connected to the same tube with the same port. In certain embodiments, each balloon is fluidly connected to its own tube with its own port. The present invention is not limited to these configurations.

As shown in FIG. 1, the tube (120) extends from the port (122) through a first balloon (110a), through a second balloon (110b), and into a third balloon (110c), wherein the tube ends.

The tube (122) features pores (125) through which fluid from the tube can enter the balloon (110). In some embodiments, the tube (122) comprises one pore (125) per balloon. In some embodiments, the tube (122) comprises two pores (125) per balloon. In some embodiments, the tube (122) comprises three pores (125) per balloon. In some embodiments, the tube (122) comprises four pores (125) per balloon. In some embodiments, the tube (122) comprises more than four pores (125) per balloon.

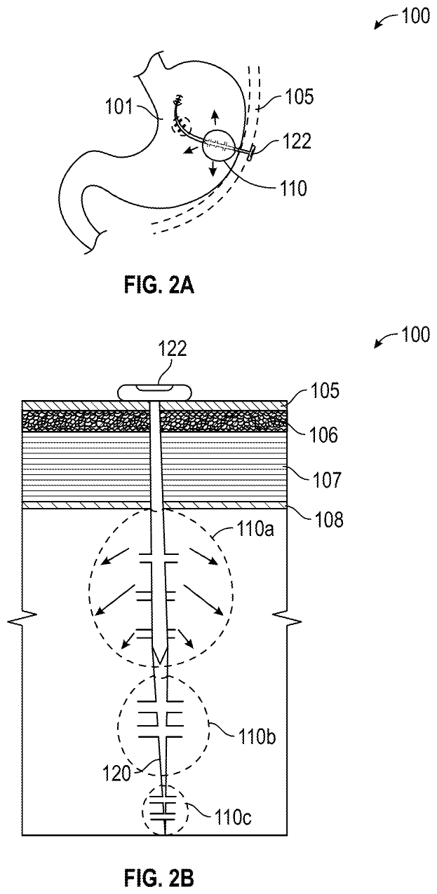

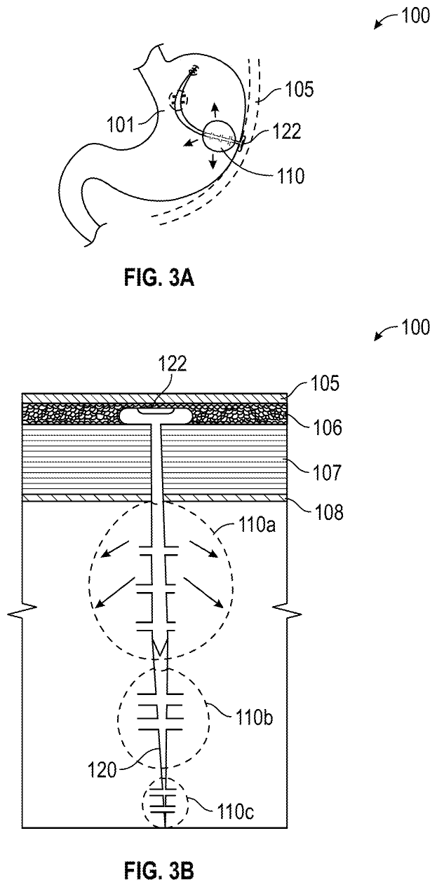

Referring to FIG. 2A and FIG. 2B, in some embodiments, the balloon system (100) is implanted such that the port (122) is outside the skin (105). The tube (120) extends through the skin (105), fat (106), muscle (107), fascia (108), and stomach (101), where the balloons (110) are placed. Referring to FIG. 3A and FIG. 3B, in some embodiments, the balloon system (100) is implanted such that the port (122) is under the skin. In the example shown in FIG. 2B, the port (122) is implanted between the fat (106) and muscle (107), where it is secured to the fascia and the tube (120) extends through the muscle (107), fascia (108), and stomach (101), where the balloons (110) are placed.

In some embodiments, the balloon system (100) of the present invention comprises three balloons, e.g., as shown in FIG. 1, FIG. 2B, and FIG. 3B, e.g., a first balloon (110a), a second balloon (110b), and a third balloon (110c). However, the present invention is not limited to three balloons. In some embodiments, the system (100) comprises one balloon. In some embodiments, the system (100) comprises two balloons. In some embodiments, the system comprises four balloons. In some embodiments, the system (100) comprises five balloons. In some embodiments, the system (100) comprises six balloons. In some embodiments, the system (100) comprises more than six balloons. In some embodiments, the system (100) comprises two, three, four, or five balloons. In some embodiments, the system (100) comprises three, four, or five balloons. In some embodiments, the system (100) comprises two or three balloons. In some embodiments, the system (100) comprises three or four balloons.

The volume of the balloons is not limited to any particular volume. Appropriate balloon volumes may be chosen based on the patient's size and/or needs. In some embodiments, the various balloons of the system may have the same volume. In some embodiments, one or more of the various balloons of the system may have a different volume. For example, in some embodiments, one or more of the balloons has a maximum volume of 15 ml. The present invention is not limited to balloons with maximum volumes of 15 ml.

In certain embodiments, one or more of the balloons of the system has a maximum volume of 5 ml. In certain embodiments, one or more of the balloons of the system has a maximum volume of 10 ml. In certain embodiments, one or more of the balloons of the system has a maximum volume of 15 ml. In certain embodiments, one or more of the balloons of the system has a maximum volume of 20 ml. In certain embodiments, one or more of the balloons of the system has a maximum volume of 25 ml. In certain embodiments, one or more of the balloons of the system has a maximum volume of 30 ml. In certain embodiments, one or more of the balloons of the system has a maximum volume of more than 30 ml.

The configuration of the balloons may be such that the first balloon is the largest and each subsequent balloon is smaller than the previous. For example, in certain embodiments, the first balloon has the largest maximum volume and the second balloon has the second largest maximum volume. In certain embodiments, the third balloon has the third largest maximum volume. In certain embodiments, the fourth balloon has the fourth largest maximum volume. In certain embodiments, the fifth balloon has the fifth largest maximum volume. In certain embodiments, the sixth balloon has the sixth largest maximum volume. Without wishing to limit the present invention to any theory or mechanism, the configuration with the sequence of balloons going from big to small may be advantageous because the larger ones would be best suited for the widest part of the stomach, whereas the smaller balloons would be best suited for where the stomach tapers. Further, the bigger balloons at the top may be better suited for activating signaling cascades telling the brain that the individual is full.

The size of the system (100) may vary depending on the particular placement approach and/or size of the patient. For example, a system that is to be implanted with the port under the skin may have a shorter neck (128) than a system that is to be implanted with the port above the skin.

The actual configuration of the system may be tailored to a patient's needs. As a non-limiting example, some patients may need a system with four balloons, the maximum volume of at least one balloon being 30 ml. Others may need only two balloons with one of the balloons having a maximum volume of 15 ml. Any number of balloons and combinations of sizes of balloons may be contemplated.

The volume of the balloons is changed by filling them with a liquid (e.g., saline) via the port (122) at the neck (128) at the end of the tube (120). As previously discussed, in certain embodiments, the balloons may be designed to fill sequentially (e.g., the first balloon first, then the second, etc.), however the present invention is not limited to sequential filling.

The system is secured in place via the port. For example, in certain embodiments, the port is secured to the skin externally. In certain embodiments, the port is implanted subcutaneously. The balloons may also provide a means of securing the system in place. For example, when the first balloon (e.g., the balloon closest to the incision through which the tube passes through to the stomach) is filled, the first balloon in that state can help anchor the system in place and prevent leakage of gastric fluid. In some embodiments, the first balloon functions as an anchor balloon and is always filled to ensure the system is held in place. For example, the first balloon may be the appropriate size to help anchor the system but not necessarily function like the subsequent balloons designed to decrease the volume of the stomach.

As previously discussed, in some embodiments, the port (122) is secured above the skin. This placement option may allow the patient to control the sizes of the balloons (110), e.g., the patient can add or release fluid from the balloons (110) via the port (122). For example, the patient may have a special occasion in which he or she wishes to release fluid from the balloons (110) for a short period of time to allow them to eat a more normal amount. In some embodiments, the port (122) is secured subcutaneously under the skin, e.g., under the skin and to the fascia. With the port (122) secured under the skin, a physician or other healthcare provider may be required to alter the balloon size.

Note that the tubing of the device may be generally flexible, e.g., not necessarily rigid. However, the present invention is not limited to flexible tubing. For example, in some embodiments, a portion of the tube is flexible and a portion of the tube is rigid.

Although there has been shown and described the preferred embodiment of the present invention, it will be readily apparent to those skilled in the art that modifications may be made thereto which do not exceed the scope of the appended claims.

Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

1. A bariatric balloon system (100) for reducing a volume of a stomach, said system (100) comprising:
a. a tube (120) having a port (122) at a first end;
b. a first balloon (110a) fluidly connected to the tube (120); and
c. a second balloon (110b) fluidly connected to the tube (120);
wherein the first balloon (110a) and the second balloon (110b) are inflatable by inserting fluid into the tube via the port (122).

2. The system (100) of claim 1, wherein the system (100) further comprises a third balloon (110c) fluidly connected to the tube (120), the third balloon (110c) is inflatable by inserting fluid into the tube (120) via the port (122).

3.-5. (canceled)

6. The system (100) of claim 1, wherein the second balloon (110b) is below the first balloon (110a) along the tube (120).

7. The system (100) of claim 2, wherein the third balloon (110c) is below the second balloon (110b) along the tube (120).

8.-10. (canceled)

11. The system (100) of claim 1, wherein the first balloon (110a) reaches its maximum volume before the second balloon (110b) reaches its maximum volume.

12. The system (100) of claim 2, wherein the second balloon (110b) reaches its maximum volume before the third balloon (110c) reaches its maximum volume.

13.-15. (canceled)

16. The system (100) of claim 1, wherein the first balloon (110a) has a maximum volume larger than that of the second balloon (110b).

17.-20. (canceled)

21. The system of claim 2, wherein the second balloon (110b) has a maximum volume larger than that of the third balloon (110c).

22.-25. (canceled)

26. The system (100) of claim 1, wherein at least a portion of the tube (120) is flexible.

27.-51. (canceled)

52. A bariatric balloon system (100) for reducing a volume of a stomach, said system (100) comprising:
a. a first tube having a first port at an external end of the first tube;
b. a first balloon (110a) fluidly connected to the first tube;
c. a second tube having a second port at an external end of the second tube; and
d. a second balloon (110b) fluidly connected to the second tube;
wherein the first balloon (110a) is inflatable by inserting fluid into the first tube via the first port, and the second balloon (110b) is inflatable by inserting fluid into the second tube via the second port.

53. The system (100) of claim 52, wherein the system (100) further comprises a third tube having a third port at an external end of the third tube, and a third balloon (110c) fluidly connected to the third tube, the third balloon (110c) is inflatable by inserting fluid into the third tube via the third port.

54.-56. (canceled)

57. The system (100) of claim 52, wherein the first balloon (110a) has a maximum volume larger than that of the second balloon (110b).

58. The system of claim 53, wherein the second balloon (110b) has a maximum volume larger than that of the third balloon (110c).

59.-62. (canceled)

63. The system (100) of claim 52, wherein at least a portion of the tube (120) is flexible.

64. A bariatric balloon system (100) for reducing a volume of a stomach, said system (100) comprising:
a. a tube (120) having a port (122) at a first end; and
b. at least a first balloon (110a) and a last balloon (110c), the tube extends through the first balloon (110a) and through the last balloon (110c), the first balloon (110a) is fluidly connected to the tube via a pore (125) disposed in the tube (120) at a location within the first balloon (110a) and the last balloon (110c) is fluidly connected to the tube (120) via a pore (125) disposed in the tube (120) at a location within the last balloon (110c);
wherein the first balloon (110a) and the last balloon (110b) are inflatable by inserting fluid into the tube via the port (122).

65. The system (100) of claim 64, wherein the system (100) comprises a first balloon (110a), a last balloon (110c), and at least one additional balloon.

66. The system (100) of claim 64, wherein the system (100) comprises a first balloon (110a), a last balloon (110c), and at least two additional balloons.

67. (canceled)

68. The system (100) of claim 64, wherein the tube (120) ends in the last balloon (110c).

69. The system (100) of claim 64, wherein the balloons fill sequentially.

70. (canceled)

71. The system (100) of claim 64, wherein at least a portion of the tube (120) is flexible.
